(19) United States
(12) Patent Application Publication
(10) Pub. No.: US 2021/0082572 A1
(43) Pub. Date: Mar. 18, 2021

(54) INFORMATION PROCESSING APPARATUS, INFORMATION PROCESSING SYSTEM, AND NON-TRANSITORY COMPUTER READABLE MEDIUM

(71) Applicant: FUJI XEROX CO., LTD., Tokyo (JP)

(72) Inventors: Hidetoshi KAKOI, Kanagawa (JP); Yasuhiro ENDO, Kanagawa (JP); Toshio FUJI, Kanagawa (JP)

(73) Assignee: FUJI XEROX CO., LTD., Tokyo (JP)

(21) Appl. No.: 16/744,277

(22) Filed: Jan. 16, 2020

(30) Foreign Application Priority Data
Sep. 18, 2019 (JP) 2019-169829

(51) Int. Cl. G16H 40/20; G16H 80/00; G16H 10/60; G06Q 10/10

(52) U.S. Cl. CPC G16H 40/20; G06Q 10/1093; G16H 10/60; G16H 80/00

(57) ABSTRACT

An information processing apparatus includes an association unit and an output control unit. The association unit associates a scheduled operation described in an operation-plan document with a scheduled operation of the same. The operation-plan document is an electronic document describing one or more scheduled operations that are to be performed during a predetermined period. The document describes one or more scheduled operations that are extracted to be performed during a period shorter than the predetermined period and one or more handwritten operation records each of which corresponds to scheduled operations described in the document. The output control unit performs control to output a scheduled operation that is associated with the scheduled operation by the association unit and an operation record that relates to the scheduled operation. The scheduled operation described in the document and the operation record are read and converted to an electronic form by an image reading apparatus.

### CROSS-REFERENCE TO RELATED APPLICATIONS

This application is based on and claims priority under 35 USC 119 from Japanese Patent Application No. 2019-169829 filed Sep. 18, 2019.

### BACKGROUND

### (i) Technical Field

The present disclosure relates to an information processing apparatus, an information processing system, and a non-transitory computer readable medium.

### (ii) Related Art

Japanese Unexamined Patent Application Publication No. 2010-231662 discloses an information processing system including a first acquisition unit, a second acquisition unit, and a management unit. When an item included in a first document whose items and the content of the items are managed by a document server is specified in an operation, the first acquisition unit acquires the content of the specified item from the document server. The second acquisition unit acquires the content of handwritten characters included in a second document in accordance with the trace of the handwritten characters. The management unit manages as the content of the specified item in the second document the content of the handwritten characters that is acquired by the second acquisition unit after the item in the first document is specified.

### SUMMARY

Aspects of non-limiting embodiments of the present disclosure relate to providing an information processing apparatus, an information processing system, and a non-transitory computer readable medium that may eliminate the inconvenience of transcribing an operation record handwritten in an operation-schedule document to an operation-plan document that is an electronic document.

Aspects of certain non-limiting embodiments of the present disclosure overcome the above disadvantages and/or other disadvantages not described above. However, aspects of the non-limiting embodiments are not required to overcome the disadvantages described above, and aspects of the non-limiting embodiments of the present disclosure may not overcome any of the disadvantages described above.

According to an aspect of the present disclosure, there is provided an information processing apparatus including an association unit and an output control unit. The association unit associates a scheduled operation described in an operation-plan document with a scheduled operation described in an operation-schedule document. The operation-plan document is an electronic document describing one or more scheduled operations that are to be performed during a predetermined period, and the operation-schedule document describes one or more scheduled operations that are extracted from the operation-plan document and that are to be performed during a period shorter than the predetermined period and one or more handwritten operation records each of which corresponds to an operation record relating to one of the one or more scheduled operations described in the operation-schedule document. In response to an instruction specifying a scheduled operation described in the operation-plan document, the output control unit performs control to output a scheduled operation that is described in the operation-schedule document and that is associated with the scheduled operation described in the operation-plan document by the association unit and an operation record that is described in the operation-schedule document and that relates to the scheduled operation described in the operation-schedule document. The scheduled operation described in the operation-schedule document and the operation record are read and converted to an electronic form by an image reading apparatus.

### BRIEF DESCRIPTION OF THE DRAWINGS

An exemplary embodiment of the present disclosure will be described in detail based on the following figures, wherein:

FIG. 1 is an explanatory diagram illustrating an overview of a care-service system;

FIG. 2 is a plan view illustrating a monthly service provision form used in an information processing system according to the exemplary embodiment of the present disclosure;

FIG. 3 is a plan view illustrating a care-service schedule sheet used in the information processing system according to the exemplary embodiment of the present disclosure;

FIG. 4 illustrates a configuration of the information processing system according to the exemplary embodiment of the present disclosure;

FIG. 5 is a block diagram illustrating a server used in the information processing system according to the exemplary embodiment of the present disclosure;

FIG. 6 is a block diagram illustrating a terminal apparatus used in the information processing system according to the exemplary embodiment of the present disclosure;

FIG. 7 is a block diagram illustrating an image forming apparatus used in the information processing system according to the exemplary embodiment of the present disclosure;

FIG. 8 is a block diagram illustrating a functional configuration of an application of the server in the information processing system according to the exemplary embodiment of the present disclosure, the functional configuration being realized when a care-service schedule sheet is output;

FIG. 9 is a flowchart illustrating an operation flow of the server in the information processing system according to the exemplary embodiment of the present disclosure, the operation flow being realized when a care-service schedule sheet is output;

FIG. 10 is a first explanatory illustration depicting an association between a monthly service provision form and a care-service schedule sheet used in the information processing system according to the exemplary embodiment of the present disclosure;

FIG. 11 is a second explanatory illustration depicting an association between a monthly service provision form and a care-service schedule sheet used in the information processing system according to the exemplary embodiment of the present disclosure;

FIG. 12 is a flowchart illustrating an operation flow of a caregiver who enters a service record on a care-service schedule sheet used in the information processing system according to the exemplary embodiment of the present disclosure;

FIG. 13 is a block diagram illustrating a functional configuration of an application of the image forming apparatus in the information processing system according to the exemplary embodiment of the present disclosure, the functional configuration being realized when a care-service schedule sheet (with service record) is submitted;

FIG. 14 is a block diagram illustrating a functional configuration of an application of the server in the information processing system according to the exemplary embodiment of the present disclosure, the functional configuration being realized when a care-service schedule sheet (with service record) is submitted;

FIG. 15 is a flowchart illustrating an operation flow of the server and the image forming apparatus in the information processing system according to the exemplary embodiment of the present disclosure, the operation flow being realized when a care-service schedule sheet (with service record) is submitted;

FIG. 16 is a table illustrating a correspondence between a monthly service provision form and a care-service schedule sheet (with service record) used in the information processing system according to the exemplary embodiment of the present disclosure;

FIG. 17 is a flowchart illustrating an operation flow of a care-service provider (office staff) in the information processing system according to the exemplary embodiment of the present disclosure, the operation flow being realized when a care-service schedule sheet (with service record) is transcribed;

FIG. 18 is a block diagram illustrating a functional configuration of an application of the server in the information processing system according to the exemplary embodiment of the present disclosure, the functional configuration being realized when a care-service schedule sheet (with service record) is transcribed; and

FIG. 19 is a sequence chart illustrating an operation flow of the server in the information processing system according to the exemplary embodiment of the present disclosure, the operation flow being realized when a care-service schedule sheet (with service record) is transcribed.

### DETAILED DESCRIPTION

Next, an exemplary embodiment of the present disclosure applied to a care service will be described.

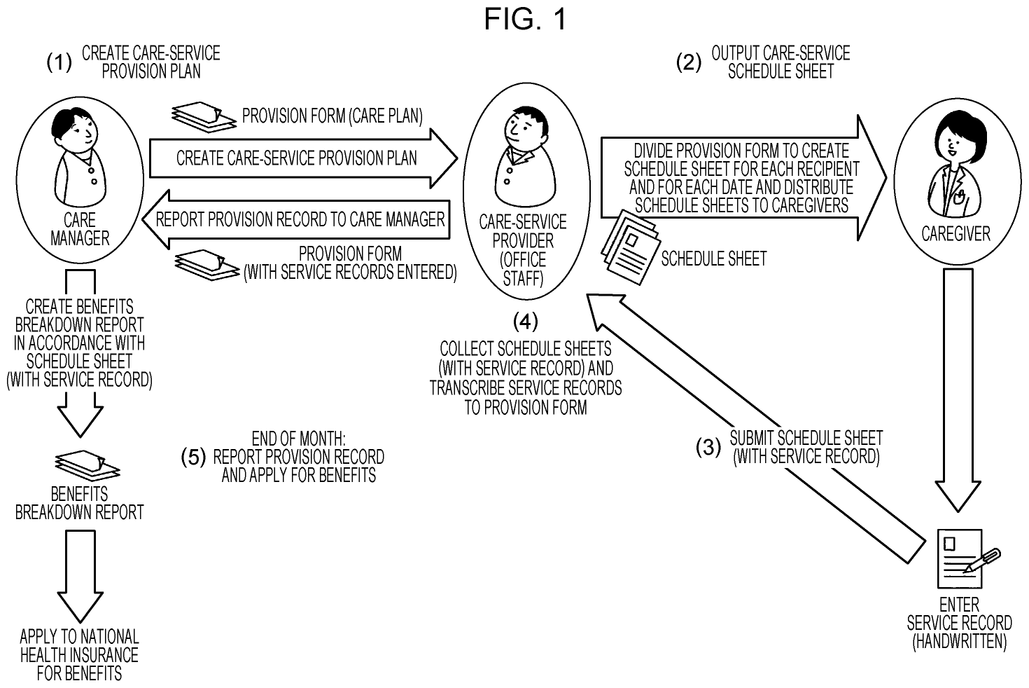

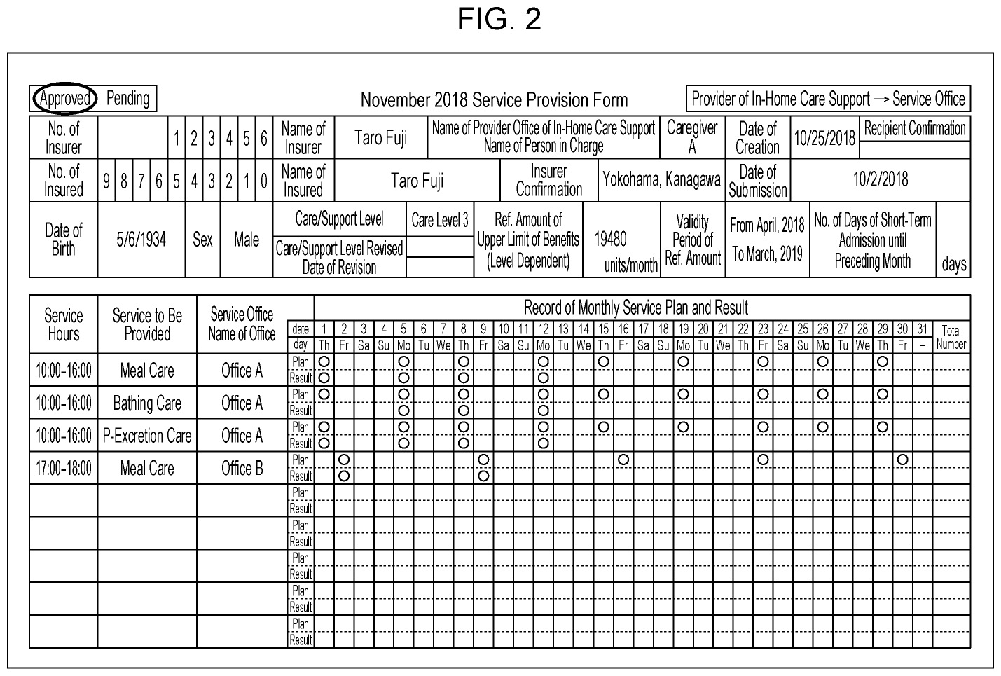

FIG. 1 illustrates an overview of a care-service system. First, a care-service provision plan is created as denoted by 1. A monthly service provision form (hereinafter referred to as a provision form) is created based on the care-service provision plan. The monthly service provision form, which is an operation-plan document, is a plan for a month and is determined in accordance with the upper limit of the amount of care benefits, and the upper limit is determined based on a care level. A care manager creates the provision form and presents the provision form to a care-service provider. The care manager refers to recipient information including the care level of a recipient when creating the provision form. As illustrated in FIG. 2, the provision form contains items such as a service to be supplied, a service provider and a service office, and scheduled dates for each recipient. The care manager is a care-support expert and has a license as a certified care worker. The care-service provider, who is an office staff, supervises at each care-service office (such as a day care center) caregivers who belong to the care-service provider and assigns care-service tasks to each of the caregivers.

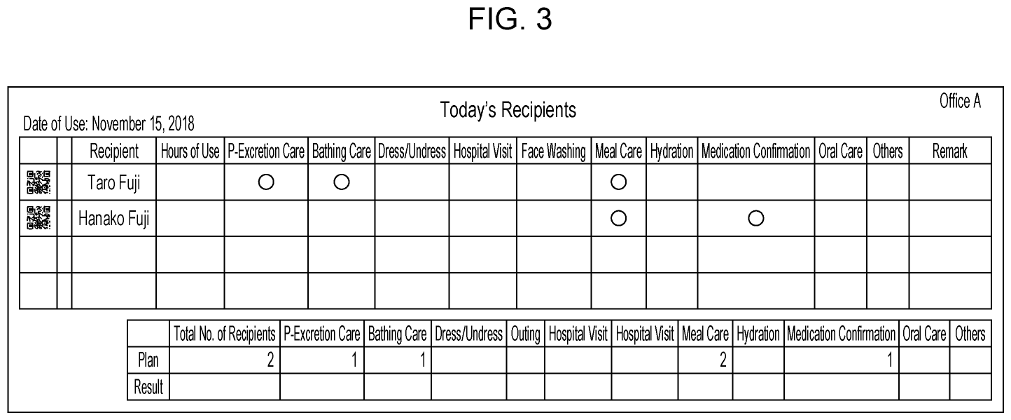

Next, a care-service schedule sheet is output as denoted by 2. The care-service schedule sheet (hereinafter referred to as a schedule sheet), which is an operation-schedule document, is used to give instructions on a daily task (service) to each caregiver. The care-service provider creates schedule sheets and distributes the schedule sheets to the caregivers. As illustrated in FIG. 3, the schedule sheet contains items such as recipients and scheduled tasks. The caregiver, who belongs to or is contracted to the care-service provider, provides a care service and takes care of a care-service recipient. The care-service recipient is certified as a person at a care level (or at a support level) by a municipality and receives a care service.

Then, a schedule sheet (with service record) is submitted as denoted by 3. The schedule sheet (with service record) is the schedule sheet distributed to the caregiver in the procedure denoted by 2, and the caregiver enters a service record of a provided care service by handwriting and submits the schedule sheet (with service record) to the care-service provider (office staff).

Subsequently, schedule sheets (with service record) are collected, and service records are transcribed to the provision form as denoted by 4. The care-service provider (office staff) transcribes service records to the provision form. The care-service provider (office staff) sends to the care manager the provision form to which service records have been transcribed, reporting a provision record to the care manager.

Then, a provision record report is submitted, and an application for benefits are filed as denoted by 5. The submission of the provision record report and the filing of the application for benefits are made, for example, at the end of a month. The care manager creates a care-benefits breakdown report. The care-benefits breakdown report is created to calculate the amount of care benefits, and the care manager submits the care-benefits breakdown report to the National Health Insurance Association Federation (National Health Insurance).

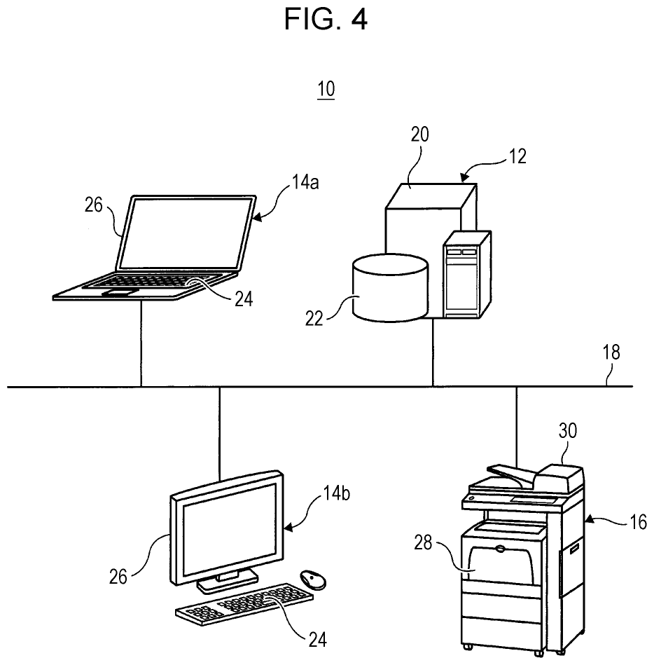

FIG. 4 illustrates an information processing system 10 according to the exemplary embodiment of the present disclosure. The information processing system 10 includes a server 12, terminal apparatuses 14a and 14b, and an image forming apparatus 16. The server 12 is an information processing apparatus, and each of the terminal apparatuses 14a and 14b is constituted by, for example, a personal computer. The server 12, the terminal apparatuses 14a and 14b, and the image forming apparatus 16 are connected to each other via a network 18.

The server 12 is constituted by a server body 20 and a database unit 22. The database unit 22 is a large-capacity storage device. Each of the terminal apparatuses 14a and 14b includes an operation device 24 and a display 26, and the operation device 24 is constituted by a keyboard and the like. The image forming apparatus 16 includes an image forming unit 28 and an image reading unit 30. The image forming unit 28 forms an image on a recording medium. The image reading unit 30 reads an image in a document and sends a read image to, for example, the server 12. The network 18 may be a local area network or the Internet.

The terminal apparatus 14a is operated by, for example, a care manager, and the server 12, the terminal apparatus 14b, and the image forming apparatus 16 are placed in a care-service office.

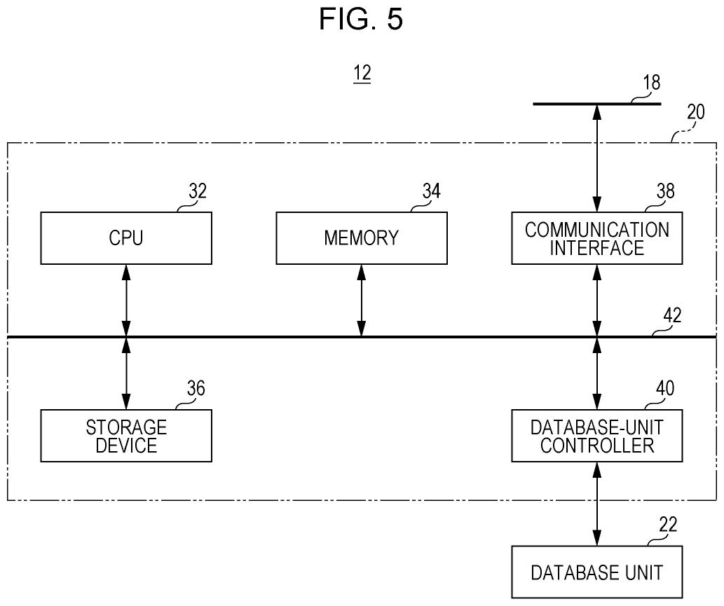

FIG. 5 is a block diagram illustrating a hardware configuration of the server 12. The server body 20 includes a central processing unit (CPU) 32, a memory 34, a storage device 36, a communication interface 38, and a database-unit controller 40. The CPU 32, the memory 34, the storage device 36, the communication interface 38, and the database-unit controller 40 are connected to each other via a bus 42.

The CPU 32 performs predetermined procedures in accordance with a control program stored in the memory 34. The storage device 36 is constituted by, for example, a hard disk and stores software and data needed for operation. The communication interface 38 inputs and outputs data via the network 18 described above. The database-unit controller 40 is configured to control data input and output to and from the database unit 22.

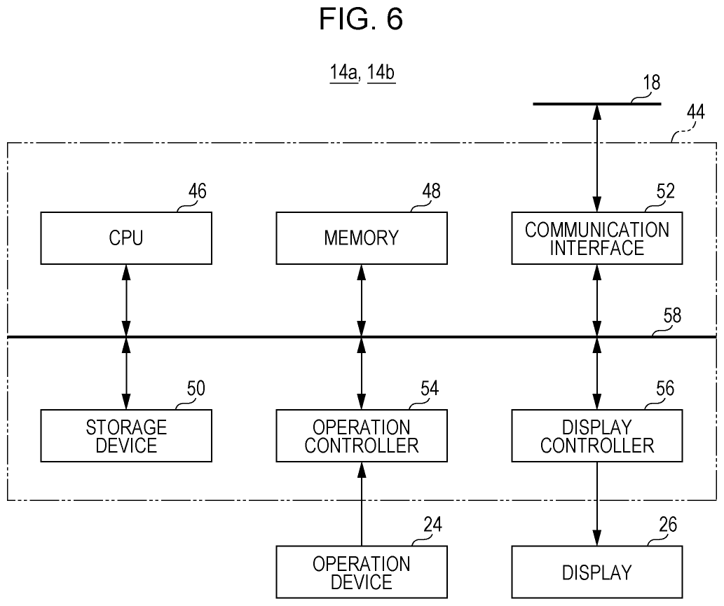

FIG. 6 is a block diagram illustrating a hardware configuration of each of the terminal apparatuses 14a and 14b. Each of the terminal apparatuses 14a and 14b includes a main controller 44. The main controller 44 includes a CPU 46, a memory 48, a storage device 50, a communication interface 52, an operation controller 54, and a display controller 56. The CPU 46, the memory 48, the storage device 50, the communication interface 52, the operation controller 54, and the display controller 56 are connected to each other via a bus 58.

The CPU 46 performs predetermined procedures in accordance with a control program stored in the memory 48. The storage device 50 is constituted by, for example, a hard disk and stores software and data needed for operation. The communication interface 52 inputs and outputs data via the network 18 described above. The operation controller 54 is connected to the operation device 24 and controls input from the operation device 24. The display controller 56 is connected to the display 26 and controls output to the display 26.

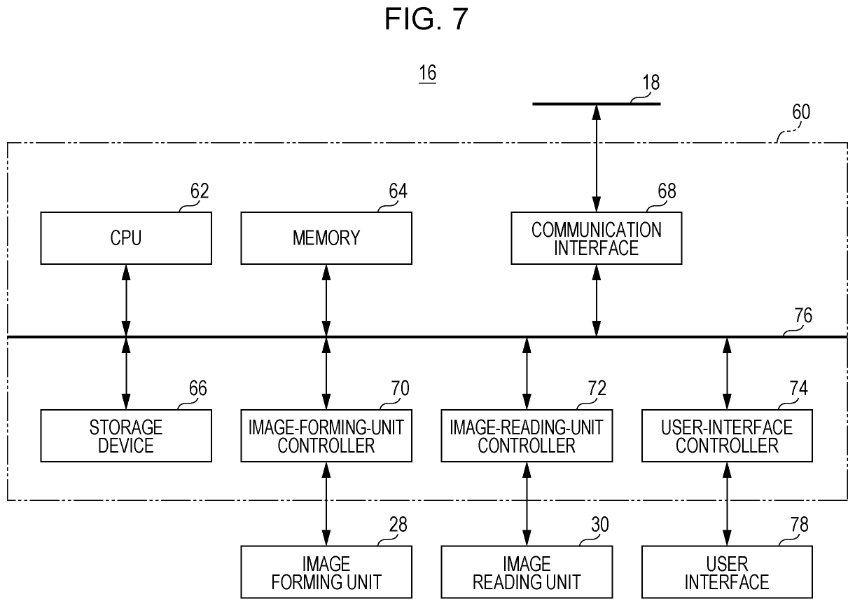

FIG. 7 is a block diagram illustrating a hardware configuration of the image forming apparatus 16. The image forming apparatus 16 includes a main controller 60. The main controller 60 includes a CPU 62, a memory 64, a storage device 66, a communication interface 68, an image-forming-unit controller 70, an image-reading-unit controller 72, and a user-interface controller 74. The CPU 62, the memory 64, the storage device 66, the communication interface 68, the image-forming-unit controller 70, the image-reading-unit controller 72, and the user-interface controller 74 are connected to each other via a bus 76.

The CPU 62 performs predetermined procedures in accordance with a control program stored in the memory 64. The storage device 66 is constituted by, for example, a hard disk and stores software and data needed for operation. The communication interface 68 inputs and outputs data via the network 18 described above. The image-forming-unit controller 70 is connected to the image forming unit 28 and controls the image forming unit 28. The image-reading-unit controller 72 is connected to the image reading unit 30 and controls the image reading unit 30. The user-interface controller 74 is connected to a user interface 78 and controls data input and output to and from the user interface 78.

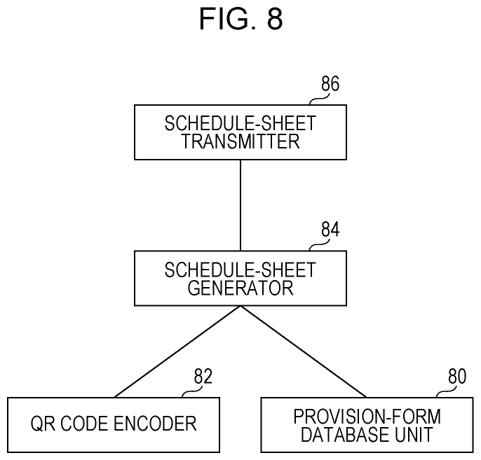

FIG. 8 illustrates a functional configuration of an application of the server 12. The functional configuration is realized when a care-service schedule sheet is output as described above and denoted by 2.

A provision-form database unit 80 stores provision forms that a care manager creates by using the terminal apparatus 14a. A QR code (registered trademark) encoder 82 generates a QR code to be attached to a schedule sheet to be created. A schedule-sheet generator 84 generates a schedule sheet corresponding to a schedule of the day and attaches a corresponding QR code to the schedule sheet. The schedule of the day is extracted from the provision-form database unit 80. A schedule-sheet transmitter 86 transmits the schedule sheet generated by the schedule-sheet generator 84 to the image forming apparatus 16. The image forming apparatus 16 causes the storage device 66 to store the transmitted schedule sheet.

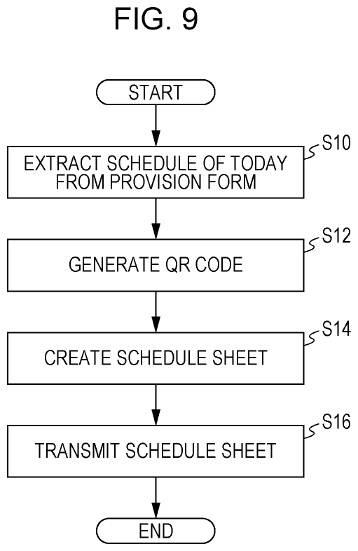

FIG. 9 is a flowchart illustrating an operation flow of the server 12. The operation flow is realized when a care-service schedule sheet is output as described above and denoted by 2.

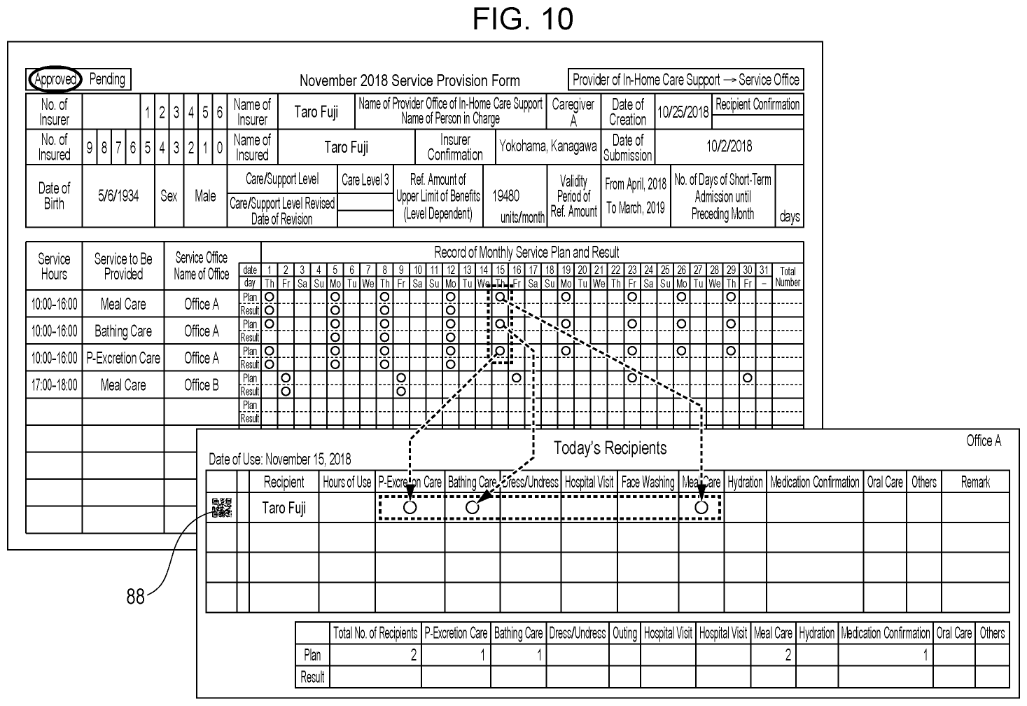

First, a schedule of the day is extracted from a provision form in step S10. Specifically, as illustrated in FIG. 10, on Nov. 15, 2018, scheduled tasks for Nov. 15, 2018 are extracted from the provision form for “Taro Fuji”. In this example schedule, the scheduled tasks are “Meal Care”, “Bathing Care”, and “P-Excretion Care”. The term “P-Excretion Care” indicates an excretion care by using a portable toilet.

Next, a QR code is generated in step S12. The file ID of the provision form (for example, the file ID for “Taro Fuji”), an office ID, a column ID, and a row ID of a schedule sheet are encoded in the QR code. An ID is a symbol to specify an object, and the office ID is a symbol to specify a combination of a provider and an office.

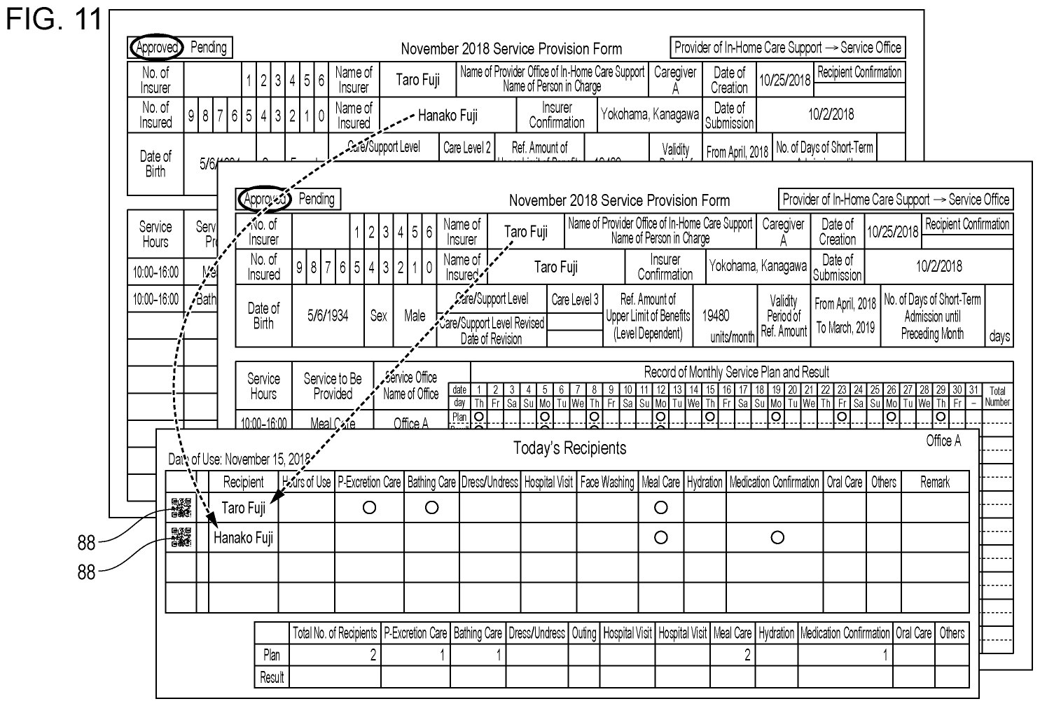

Then, a schedule sheet is created in step S14. The schedule sheet contains rows corresponding to provision forms in advance, and scheduled tasks are presented in a row for each recipient. In this example, the first row is allocated to a recipient “Taro Fuji”, and “Meal Care”, “Bathing Care”, and “P-Excretion Care” are transcribed as the scheduled tasks. A QR code 88 is attached to this row. The schedule sheet may contain a plurality of recipients. As illustrated in FIG. 11, a schedule for the day may be extracted from the file for “Hanako Fuji”, and the scheduled tasks may be transcribed into the second row of the schedule sheet. The QR code 88 generated in step S12 is attached to a corresponding row of this schedule sheet.

Next, the schedule sheet created in step S14 is transmitted to the image forming apparatus 16 in step S16, and the process ends.

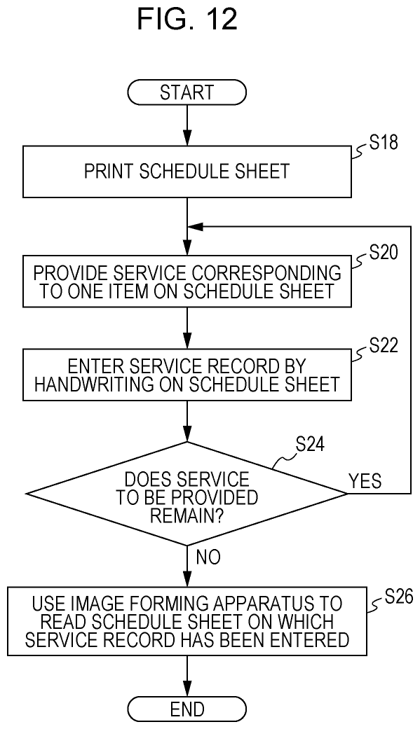

FIG. 12 is a flowchart illustrating an operation flow of a caregiver who enters a service record on a schedule sheet as described above and denoted by 3.

First, the caregiver prints a schedule sheet by using the image forming apparatus 16 in step S18. The schedule sheets are stored in the storage device 66 of the image forming apparatus 16 separately for each caregiver, and a schedule sheet is printed, for example, when the caregiver holds the ID card of the caregiver toward the image forming apparatus 16 and provides an instruction for printing. As described above, a QR code is embedded in each row of the schedule sheet, and the file ID of a provision form, an office ID, and a column ID, and a row ID of the schedule sheet are encoded.

Then, the caregiver provides a service corresponding to an item on the schedule sheet in step S20. Next, the caregiver enters a service record by handwriting on the schedule sheet in step S22 after providing the service corresponding to the item. Since the service record may be entered in free format, a schedule box is checked or a word such as “completed” or “finished” is entered. If the service is not provided, a word such as “canceled” or “aborted” is entered. In addition, a health condition of the recipient may be entered.

Next, it is determined in step S24 whether a service to be provided remains. If it is determined in step S24 that a service to be provided remains, the process returns to step S20, and the remaining service is provided. Then, a service record concerning the remaining service is entered on the schedule sheet in step S22. If it is determined in step S24 that no service to be provided remains, the process proceeds to step S26. In step S26, the caregiver (or the care-service provider) uses the image forming apparatus 16 to read the schedule sheet on which the service record has been entered, and the process ends.

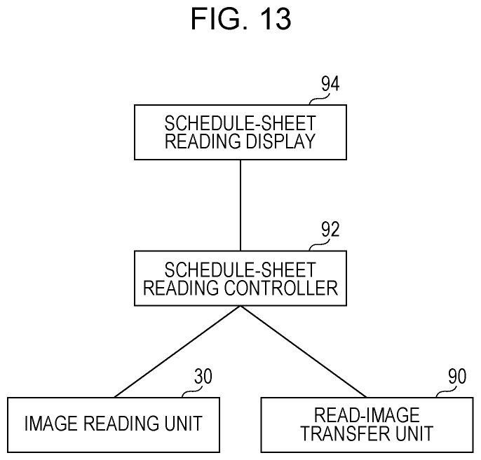

FIG. 13 illustrates a functional configuration of an application of the image forming apparatus 16. The functional configuration is realized when a schedule sheet (with service record) is submitted as described above and denoted by 3.

The image reading unit 30 reads a schedule sheet (with service record). A read-image transfer unit 90 transfers an image read by the image reading unit 30 to the server 12 via the network 18. A schedule-sheet reading controller 92 controls the image reading unit 30 and the read-image transfer unit 90. The schedule-sheet reading controller 92 performs control so as to cause a schedule-sheet reading display 94 to display the image read by the image reading unit 30.

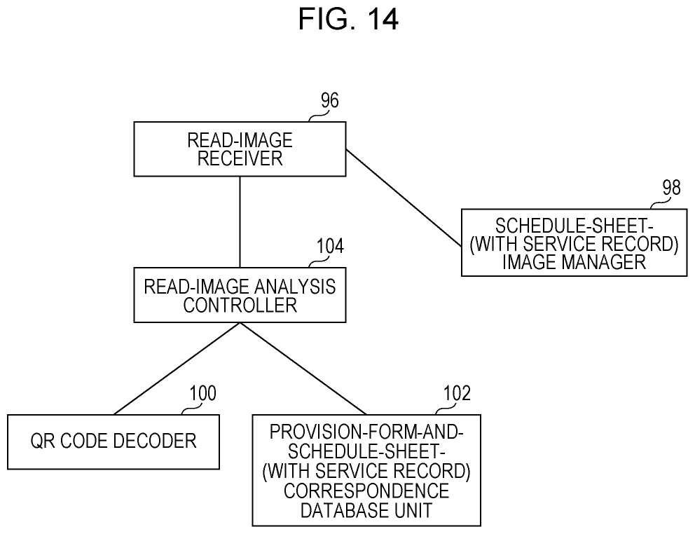

FIG. 14 illustrates a functional configuration of an application of the server 12. The functional configuration is realized when a schedule sheet (with service record) is submitted as described above and denoted by 3.

A read-image receiver 96 receives the schedule sheet (with service record), which is transmitted by the image forming apparatus 16. A schedule-sheet-(with service record) image manager 98 stores and manages the schedule sheet (with service record), which is received by the read-image receiver 96. A QR code decoder 100 decodes the QR code and obtains the original data. A provision-form-and-schedule-sheet-(with service record) correspondence database unit 102 is formed inside the database unit 22 described above. A read-image analysis controller 104 analyzes the QR code on the received schedule sheet (with service record) and performs control so as to associate a provision form with data extracted from the schedule sheet (with service record) and store the associated data in the provision-form-and-schedule-sheet-(with service record) correspondence database unit 102.

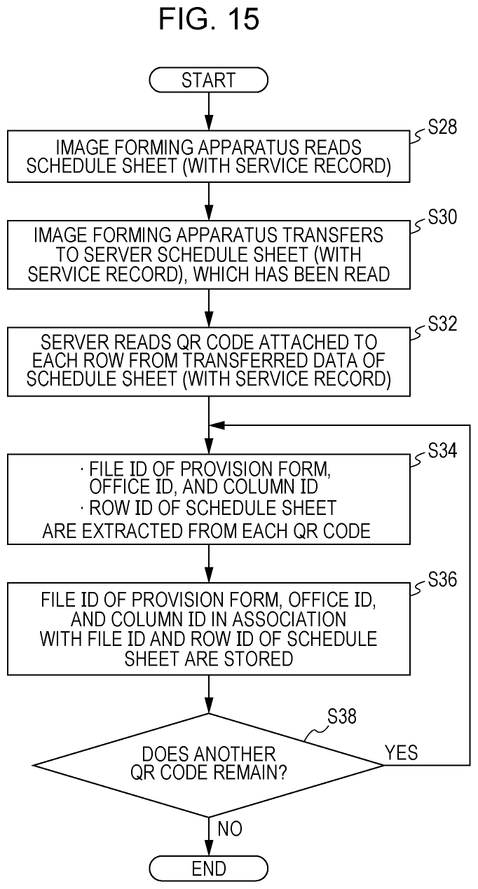

FIG. 15 is a flowchart illustrating an operation flow of the server 12 and the image forming apparatus 16. The operation flow is realized when a schedule sheet (with service record) is submitted as described above and denoted by 3.

First, the image forming apparatus 16 reads the schedule sheet (with service record) in step S28. Next, in step S30, the image forming apparatus 16 transfers to the server 12 the schedule sheet (with service record), which has been read in step S28.

Then, in step S32, the server 12 reads a QR code attached to each row from the transferred data of the schedule sheet (with service record). Next, the file ID of a provision form, an office ID, a column ID, and a row ID of the schedule sheet are extracted from each QR code in step S34.

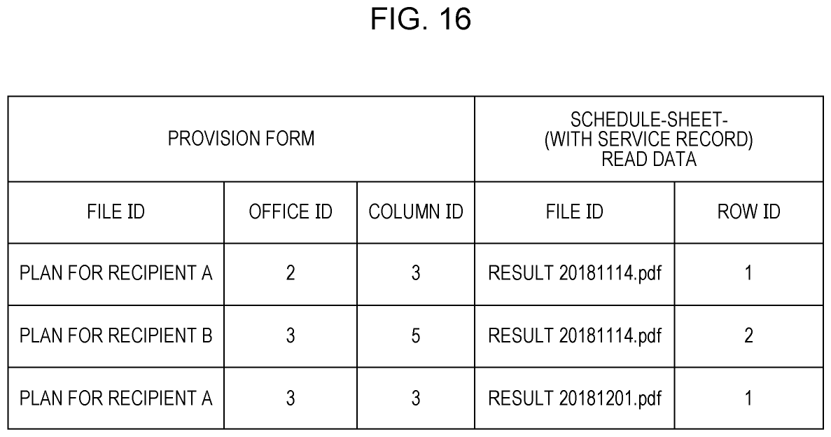

Next, in step S36, as illustrated in FIG. 16, the file ID of the provision form, the office ID, and the column ID are associated with the file ID and the row ID of the schedule sheet (with service record). The file ID of the schedule sheet (with service record) is an ID that is assigned when the schedule sheet (with service record) is read and a symbol to specify the schedule sheet (with service record) that is read.

Next, it is determined in step S38 whether another QR code remains. If it is determined in step S38 that another QR code remains, the process returns to step S34. If it is determined in step S38 that no QR code remains, the process ends.

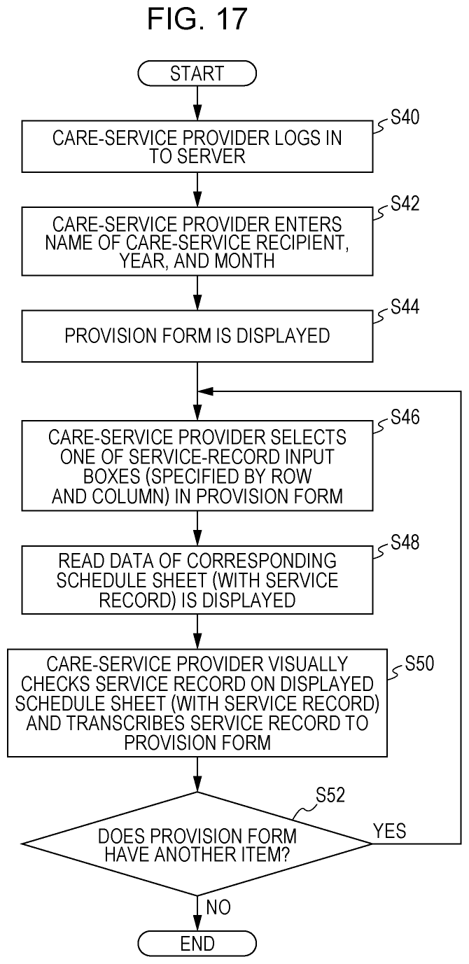

FIG. 17 is a flowchart illustrating an operation flow of a care-service provider (office staff). The operation flow is realized when a schedule sheet (with service record) is transcribed as described above and denoted by 4.

First, the care-service provider (office staff) enters, for example, an ID and a password and logs in to the server 12 in step S40. Next, the care-service provider (office staff) enters the name of a care-service recipient and the year and the month of a required provision form in step S42.

Then, in step S44, the server 12 performs control so that the corresponding provision form is displayed, causing the display 26 of the terminal apparatus 14b of the care-service provider (office staff) to display the provision form.

Subsequently, the care-service provider (office staff) selects one of service-record input boxes (specified by a row and a column) in the provision form in step S46. A service-record input box is used to receive a service record. For example, a portion of an area enclosed by dotted rectangular lines in the provision form illustrated in FIG. 10 is selected with an action such as a click.

Then, the server 12 performs control so that the corresponding schedule sheet (with service record) is displayed in step S48, for example, as illustrated in FIG. 10, causing the display 26 of the terminal apparatus 14b of the care-service provider (office staff) to display the corresponding schedule sheet (with service record). Specifically, the selected service-record input box provides the file ID of the provision form, an office ID, and a column ID, which are stored in the provision-form-and-schedule-sheet-(with service record) correspondence database unit 102. The provision-form-and-schedule-sheet-(with service record) correspondence database unit 102 is searched by using the file ID of the provision form, the office ID, and the column ID, which are determined in this way, to find the file ID of the corresponding schedule sheet (with service record), and the schedule sheet (with service record) having the file ID that has been found is displayed. If a plurality of caregivers are involved, a plurality of schedule sheets (with service record) are displayed.

Since details are hard to recognize when the entire page of a schedule sheet (with service record) is displayed, a target region may be, for example, enclosed with a red boundary or clipped to assist transcription. The care-service provider (office staff) may be allowed to manually change the way to display a target region. In addition, if an item written outside the service-record input boxes is detected, the display region may be expanded to encompass the item.

Next, the care-service provider (office staff) visually checks the service record on the displayed schedule sheet (with service record) and transcribes the service record to one or more service-record input boxes in the provision form in step S50. However, if the schedule sheet (with service record) has a fixed format, a service record that has been read may be transcribed automatically instead of manually.

Next, it is determined in step S52 whether the provision form has another item whose service record is to be entered. If it is determined in step S52 that the provision form has another item, the process returns to step S46. If it is determined in step S52 that the provision form has no other item, the process ends.

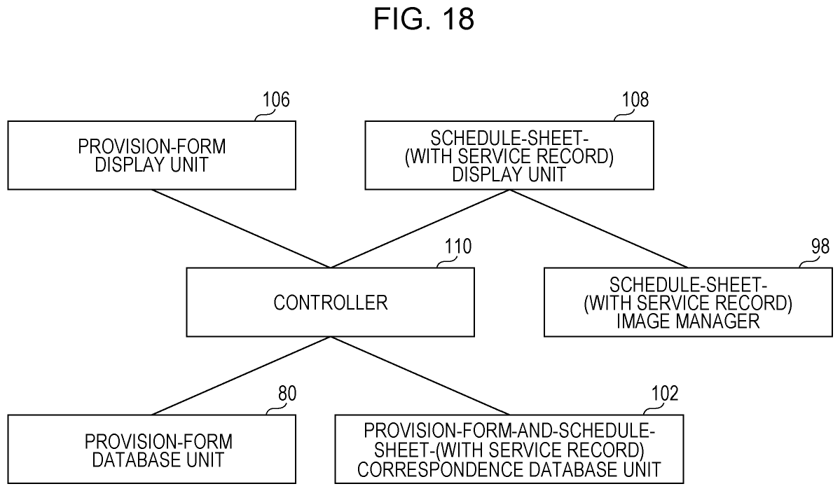

FIG. 18 illustrates a functional configuration of an application of the server 12. The functional configuration is realized when a schedule sheet (with service record) is transcribed as described above and denoted by 4.

As described above, the provision-form database unit 80 stores provision forms. The schedule-sheet-(with service record) image manager 98 stores and manages schedule sheets (with service record). The provision-form-and-schedule-sheet-(with service record) correspondence database unit 102 stores information that associates provision forms with schedule sheets (with service record). A provision-form display unit 106 performs control so that provision forms are displayed. A schedule-sheet-(with service record) display unit 108 performs control so that schedule sheets (with service record) are displayed. A controller 110 controls the provision-form database unit 80, the provision-form-and-schedule-sheet-(with service record) correspondence database unit 102, the provision-form display unit 106, and the schedule-sheet-(with service record) display unit 108.

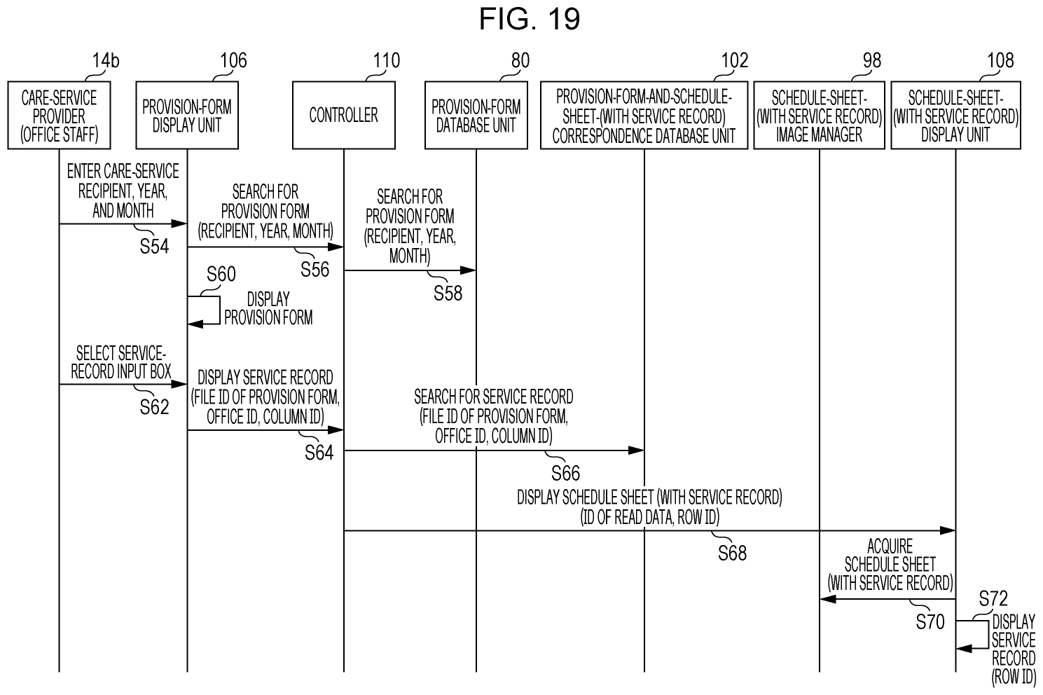

FIG. 19 is a sequence chart illustrating an operation flow of the server 12. The operation flow is realized when a schedule sheet (with service record) is transcribed as described above and denoted by 4.

First, data concerning a care-service recipient, the year, and the month is entered into the provision-form display unit 106 from the terminal apparatus 14b of the care-service provider (office staff) in step S54. Then, the data concerning a care-service recipient, the year, and the month that has been entered is sent to the controller 110 in step S56. The controller 110 sends search data to the provision-form database unit 80 and performs search in step S58. The provision-form display unit 106 performs control so that a found provision form is displayed by the terminal apparatus 14b of the care-service provider (office staff) in step S60, causing the terminal apparatus 14b to display the found provision form.

Selection information concerning the service-record input boxes is entered into the provision-form display unit 106 from the terminal apparatus 14b of the care-service provider (office staff) in step S62. Then, the selection information, which has been entered, concerning the service-record input boxes is sent to the controller 110 in step S64. Next, the controller 110 sends search information for a service record (the file ID of the provision form, an office ID, and a column ID) to the provision-form-and-schedule-sheet-(with service record) correspondence database unit 102 in step S66. In step S68, the controller 110 sends record-indication information (the file ID of a schedule sheet (with service record) and a row ID) to the schedule-sheet-(with service record) display unit 108 in accordance with the search result. Next, in step S70, the schedule-sheet-(with service record) display unit 108 provides the schedule-sheet-(with service record) image manager 98 with an instruction to acquire a schedule sheet (with service record). Subsequently, the schedule-sheet-(with service record) display unit 108 performs control so that the service record is displayed based on the row ID in step S72, causing the terminal apparatus 14b to display the service record contained in the schedule sheet.

An example in which the present disclosure is applied to a care-service system is described in the above exemplary embodiment. The present disclosure is not limited to the above example and can be applied to, for example, a case where a machine is inspected in a facility such as a plant. Specifically, the present disclosure can be applied to a case where, when a machine is inspected, a schedule sheet is created by extracting from a provision form one or more items to be checked and a service record is entered on the schedule sheet.

The foregoing description of the exemplary embodiment of the present disclosure has been provided for the purposes of illustration and description. It is not intended to be exhaustive or to limit the disclosure to the precise forms disclosed. Obviously, many modifications and variations will be apparent to practitioners skilled in the art. The embodiment was chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. An information processing apparatus comprising:
an association unit that associates a scheduled operation described in an operation-plan document with a scheduled operation described in an operation-schedule document, the operation-plan document being an electronic document describing one or more scheduled operations that are to be performed during a predetermined period, the operation-schedule document describing one or more scheduled operations that are extracted from the operation-plan document and that are to be performed during a period shorter than the predetermined period and one or more handwritten operation records each of which corresponds to an operation record relating to one of the one or more scheduled operations described in the operation-schedule document; and
an output control unit that, in response to an instruction specifying a scheduled operation described in the operation-plan document, performs control to output a scheduled operation that is described in the operation-schedule document and that is associated with the scheduled operation described in the operation-plan document by the association unit and an operation record that is described in the operation-schedule document and that relates to the scheduled operation described in the operation-schedule document, the scheduled operation described in the operation-schedule document and the operation record being read and converted to an electronic form by an image reading apparatus.

2. The information processing apparatus according to claim 1,
wherein the association unit associates the scheduled operation described in the operation-plan document with the scheduled operation described in the operation-schedule document by attaching coded information to the operation-schedule document.

3. The information processing apparatus according to claim 1,
wherein the operation-plan document contains one or more scheduled tasks as the one or more scheduled operations and the output control unit performs control to output the scheduled operation described in the operation-schedule document and the operation record when at least one of the one or more scheduled tasks contained in the operation-plan document is specified.

4. The information processing apparatus according to claim 2,
wherein the operation-plan document contains one or more scheduled tasks as the one or more scheduled operations and the output control unit performs control to output the scheduled operation described in the operation-schedule document and the operation record when at least one of the one or more scheduled tasks contained in the operation-plan document is specified.

5. The information processing apparatus according to claim 1,
wherein the operation-plan document contains one or more scheduled dates as the one or more scheduled operations and the output control unit performs control to output the scheduled operation described in the operation-schedule document and the operation record when at least one of the one or more scheduled dates contained in the operation-plan document is specified.

6. The information processing apparatus according to claim 2,
wherein the operation-plan document contains one or more scheduled dates as the one or more scheduled operations and the output control unit performs control to output the scheduled operation described in the operation-schedule document and the operation record when at least one of the one or more scheduled dates contained in the operation-plan document is specified.

7. The information processing apparatus according to claim 1,
wherein the operation-plan document contains one or more scheduled locations as the one or more scheduled operations and the output control unit performs control to output the scheduled operation described in the operation-schedule document and the operation record when at least one of the one or more scheduled locations contained in the operation-plan document is specified.

8. The information processing apparatus according to claim 2,
wherein the operation-plan document contains one or more scheduled locations as the one or more scheduled operations and the output control unit performs control to output the scheduled operation described in the operation-schedule document and the operation record when at least one of the one or more scheduled locations contained in the operation-plan document is specified.

9. The information processing apparatus according to claim 1,
wherein the operation-plan document is a list that contains one or more scheduled tasks and one or more scheduled dates as the one or more scheduled operations and that assigns one of the one or more scheduled dates to each of the one or more scheduled tasks, and the output control unit performs control to output the scheduled operation described in the operation-schedule document and the operation record when at least one of the one or more scheduled tasks and at least one of the one or more scheduled dates are specified.

10. The information processing apparatus according to claim 2,
wherein the operation-plan document is a list that contains one or more scheduled tasks and one or more scheduled dates as the one or more scheduled operations and that assigns one of the one or more scheduled dates to each of the one or more scheduled tasks, and the output control unit performs control to output the scheduled operation described in the operation-schedule document and the operation record when at least one of the one or more scheduled tasks and at least one of the one or more scheduled dates are specified.

11. The information processing apparatus according to claim 1,
wherein the operation-plan document is a list that contains one or more scheduled locations and one or more scheduled dates as the one or more scheduled operations and that assigns one of the one or more scheduled dates to each of the one or more scheduled locations, and the output control unit performs control to output the scheduled operation described in the operation-schedule document and the operation record when at least one of the one or more scheduled locations and at least one of the one or more scheduled dates are specified.

12. The information processing apparatus according to claim 2,
wherein the operation-plan document is a list that contains one or more scheduled locations and one or more scheduled dates as the one or more scheduled operations and that assigns one of the one or more scheduled dates to each of the one or more scheduled locations, and the output control unit performs control to output the scheduled operation described in the operation-schedule document and the operation record when at least one of the one or more scheduled locations and at least one of the one or more scheduled dates are specified.

13. The information processing apparatus according to claim 1,
wherein the operation-plan document is a monthly service provision form to provide care service and the operation-schedule document is a schedule sheet for daily use of care service performed by one or more caregivers.

14. An information processing system comprising:
an image reading apparatus that reads an image; and
an information processing apparatus that processes information acquired from an image that is read by the image reading apparatus,
wherein the information processing apparatus includes
an association unit that associates a scheduled operation described in an operation-plan document with a scheduled operation described in an operation-schedule document, the operation-plan document being an electronic document describing one or more scheduled operations that are to be performed during a predetermined period, the operation-schedule document describing one or more scheduled operations that are extracted from the operation-plan document and that are to be performed during a period shorter than the predetermined period and one or more handwritten operation records each of which corresponds to an operation record relating to one of the one or more scheduled operations described in the operation-schedule document, and
an output control unit that, in response to an instruction specifying a scheduled operation described in the operation-plan document, performs control to output a scheduled operation that is described in the operation-schedule document and that is associated with the scheduled operation described in the operation-plan document by the association unit and an operation record that is described in the operation-schedule document and that relates to the scheduled operation described in the operation-schedule document, the scheduled operation described in the operation-schedule document and the operation record being read and converted to an electronic form by an image reading apparatus.

15. A non-transitory computer readable medium storing a program causing a computer to execute a process for image processing, the process comprising:
associating a scheduled operation described in an operation-plan document with a scheduled operation described in an operation-schedule document, the operation-plan document being an electronic document describing one or more scheduled operations that are to be performed during a predetermined period, the operation-schedule document describing one or more scheduled operations that are extracted from the operation-plan document and that are to be performed during a period shorter than the predetermined period and one or more handwritten operation records each of which corresponds to an operation record relating to one of the one or more scheduled operations described in the operation-schedule document; and
in response to an instruction specifying a scheduled operation described in the operation-plan document, performing control to output a scheduled operation that is described in the operation-schedule document and that is associated with the scheduled operation described in the operation-plan document and an operation record that is described in the operation-schedule document and that relates to the scheduled operation described in the operation-schedule document, the scheduled operation described in the operation-schedule document and the operation record being read and converted to an electronic form by an image reading apparatus.
